(19) United States
(12) Patent Application Publication
(10) Pub. No.: US 2015/0051557 A1
(43) Pub. Date: Feb. 19, 2015

(54) DEVICES AND METHODS OF ADMINISTERING OPTHALMIC MEDICATIONS

(71) Applicant: Duke University, Durham, NC (US)

(72) Inventors: Kelly Muir, Durham, NC (US); David L. Foshee, Apex, NC (US); Robert C. Hall, Apex, NC (US); Kristin Benokraitis, Durham, NC (US); Theodore J. Mosler, Raleigh, NC (US); David Epstein, Bahama, NC (US)

(21) Appl. No.: 14/386,516

(22) PCT Filed: Mar. 20, 2013
(86) PCT No.: PCT/US13/33163

§ 371 Date: Sep. 19, 2014

Related U.S. Application Data
(60) Provisional application No. 61/613,164, filed on Mar. 20, 2012.
(60) Provisional application No. 61/623,856, filed on Apr. 13, 2012.

(51) Int. Cl. A61F 9/00

(52) U.S. Cl. CPC A61F 9/0026
USPC 604/290; 604/294

(57) ABSTRACT

A system for administering medication to an eye. The system includes a siphon tube in fluid communication with a medication reservoir, a dispensing nozzle connected to the siphon tube, and a shield coupled to the dispensing nozzle for movement relative thereto between an opened position and a closed position. The siphon tube defines a longitudinal axis extending along a substantially vertical direction when the siphon tube is in an upright position. The dispensing nozzle extends at an angle offset from the longitudinal axis when the shield is in the opened position. The nozzle and the angle are so dimensioned as to facilitate accessing the eye when the siphon tube is substantially in the upright position.

### CROSS-REFERENCE TO RELATED APPLICATIONS

This application claims the benefit of U.S. Provisional Patent Application No. 61/613,164, filed on Mar. 20, 2012, and U.S. Provisional Patent Application No. 61/623,856, filed on Apr. 13, 2012. The entire contents of each application are incorporated herein by reference.

### BACKGROUND OF THE INVENTION

Glaucoma is a chronic disease and the treatment depends largely on patient self-management with eye drops. Based on the 2000 census data, it is estimated that 2.22 million Americans suffer from open angle glaucoma, the most common form of glaucoma. Glaucoma is a leading cause of irreversible blindness worldwide, and may disproportionately affect the elderly and African-Americans in both prevalence and severity. The number of Americans with glaucoma is expected to increase by approximately 50% in the next fifteen years. It has been shown that with effective treatment, much glaucomatous vision loss can be prevented through reduction in intraocular pressure. Despite this, however, glaucoma medications often are not taken as prescribed.

The term adherence is used to describe how well the way in which a patient is actually taking a medication coincides with the advice of the health care provider. Several factors have been found to contribute to nonadherence including more frequent and complex dosing, situational factors such as competing activities, and forgetfulness. Patients also report technical difficulties with instilling eye drops, including difficulty squeezing the bottle and inability to recline the head due to arthritis. In fact, when a device was used to measure the ability of patients to squeeze a traditional eye drop bottle, 14% of subjects were unable to generate enough force to express a drop.

For patients with glaucoma, proper medication adherence includes several elements, including the ability to properly instill an eye drop into the eye and do so at least once a day and often more, for many years. Unfortunately, even experienced eye drop users are unable to self-administer eye drops without missing the eye or contaminating the bottle only about 30% of the time.

A recent study examined more than 200 glaucoma patients with low vision, 94% of whom reported taking prescription eye drops for at least 6 months. Under observation in the clinic, only 71% of these patients with glaucoma were able to instill even one drop into the eye and only 39% did so without contaminating the bottle tip by touching it to the skin or ocular surface. The investigators also queried the subjects as to their perceived problems with drop instillation. Of the subjects who reported not touching the bottle tip to the eye during drop instillation, almost one quarter contaminated the bottle tip when observed in the study setting. It is mainly this population, patients with glaucoma and vision loss, who may most need proper medication adherence to prevent progression to blindness, yet with the currently available medication delivery system, more than a quarter may not be receiving medication at all and more than half are contaminating the bottle in the process.

### SUMMARY OF THE INVENTION

Currently available eye drop bottles require inversion (see, for example, FIG. 1), which poses difficulty for many patients. In order to dispense a drop from a traditional eye drop bottle, the bottle must be inverted perpendicular to the horizontal plane, so that the drop can fall downward and onto the cornea or into the cul-de-sac. Such a design requires that the patient either recline the head or lie flat such that the plane of the cornea is horizontal. As described previously, glaucoma patients with arthritis report difficulty with this positioning.

The containers and methods described herein provide a novel eye drop bottle for delivery of opthalmic medications. The present invention has the potential to improve medication adherence and reduce vision loss for patients with glaucoma.

The present invention provides delivery containers, such as eye drop bottles, which make it easier for patients to instill eye drops. The use of such containers may improve the visual outcomes of patients with glaucoma by reducing barriers to medication nonadherence.

One aspect of the present invention provides a novel eye drop bottle which does not require the patient to recline the head or squeeze the bottle to express the drop, as is required by traditional eye drop bottles.

The present invention provides an upright bottle design that ameliorates the difficulties encountered with the currently available inversion bottle approach. In one embodiment, the bottle uses a “dip tube” extending into the bottle. To administer the drop, the user unscrews the cap and rotates the dispensing tube into position. The dispensing tube is angulated and allows the user to express a drop, by squeezing the bottle, into the inferior fornix while maintaining the head in an upright position (FIG. 2).

In another embodiment, the present invention provides a modified upright eye dropper with metered delivery, drop projection, distance control and contamination protection. To use such a device (see, e.g., FIG. 3), the user flips open the cover, creating a cheek rest. This action triggers the button the user will press to deliver the drop into a “ready for delivery” state. When the user presses the button, an internal component under spring tension is released, applying pressure to a compressible chamber. This pressure acts as positive displacement, forcing medication to travel through a tube.

FIG. 2 shows a section view of an eye dropper according to an embodiment of the present invention comprising means to deliver medication in an upright orientation, access to the medication reservoir via a diptube, a pump mechanism comprising a lower inlet one-way valve, a transit tubing section of certain diameter, a compressible tubing section of larger diameter, a transit tubing section of a certain diameter, an upper exit one-way valve, and a nozzle, which may be oriented in regard to the cheekrest and the axis of the device to provide an advantageous angle of instilling a drop into the user's eye, a cheekiest that in one orientation provides a cover for the nozzle, in a second orientation provides a stop for locating the device a set distance from the eye in a supported fashion, and in transition between these states cocks an activation mechanism consisting of an extension spring for storing the activation energy, an activation arm for acting on the compressible tubing section and a trigger mechanism for selectively releasing the activation arm.

Benefits of such a design are numerous, and may include: (1) the minimization of required hand strength and dexterity; (2) greater ease for the user to hold the bottle still when delivering the eye drop; (3) providing quick eye drop delivery, minimizing the opportunity for blinking; and (4) putting the user in control of drop delivery. In an alternative construction, the button is replaced with a handle or lever as illustrated in FIG. 4.

Because the “button” is automatically triggered and spring-loaded, pressure applied to the compressible chamber is consistent and not dependent on force exerted by the patient. The consistent pressure applied to the compressible chamber positively displaces the medication volume up the tube and out the tip. Each press of the button results in a single drop, reducing the opportunity for “wasted” drops and over or under dosing.

The eye dropper as provided herein is designed to have an angled dispense tip, projecting the eye drop at an angle. In addition to the angled design, the medication fluid path (tubing) is designed to step from a larger diameter tube down to s smaller diameter tube, giving the fluid extra velocity to project outward when the compressible chamber is compressed.

The cover of the container has two purposes: (1) to protect the tip from potential contamination and (2) to act as an integral cheek rest. The cover may be made of a conforming medical grade material such as a silicone or thermoplastic elastomer for compatibility to a range of facial structures. The material can allow for the cheek rest to be comfortable, but also give the patient something to stabilize the eye dropper and maintain the proper distance from the eye during eye drop delivery. The cover can remain connected to the bottle and therefore will not be lost or thrown away. When in the “cheek rest” position, the cover can minimize potential contamination caused when the dispense tip touches facial skin or the ocular surface.

Alternative embodiments could include a reservoir configured to be above the inlet valve, eliminating the need for a dip-tube. In any configuration, the reservoir could include an inlet vent or valve to prevent negative pressure from interrupting or preventing the flow of medication out of the reservoir. The inlet vent or valve could be configured with a filter to prevent contamination of the reservoir by airborne contaminants and to prevent the medication from leaking out. Alternative mechanisms to move the fluid medication through the system may be used, for example a pump comprising a rigid cylinder and housing could replace the compressible tubing section to work in conjunction with one way valves.

In one aspect, the invention provides a system for administering medication to an eye. The system includes a siphon tube in fluid communication with a medication reservoir, a dispensing nozzle connected to the siphon tube, and a shield coupled to the dispensing nozzle for movement relative thereto between an opened position and a closed position. The siphon tube defines a longitudinal axis extending along a substantially vertical direction when the siphon tube is in an upright position. The dispensing nozzle extends at an angle offset from the longitudinal axis when the shield is in the opened position. The nozzle and the angle are so dimensioned as to facilitate accessing the eye when the siphon tube is substantially in the upright position.

In another aspect, the invention provides a system for administering medication to an eye. The system includes a container, a siphon tube in fluid communication with the container, a dispensing nozzle connected to the siphon tube, and a shield covered to the dispensing nozzle for movement relative thereto between an opened position and a closed position. The container has a base and an opening positioned substantially above the base when the container is in an upright position. The siphon tube defines a longitudinal axis. The dispensing nozzle extends at an angle offset from the longitudinal axis when the shield in the opened position. The nozzle and the angle are so dimensioned as to facilitate accessing the eye when the container is substantially in the upright position.

In another aspect, the invention provides a method for administering medication to an eye of a user. A shield coupled to a dispensing nozzle of a system for administering medication is opened, the shield is placed against a facial structure of the user, a dosing pump is activated, and the user's eye is contacted with the medication, all when the system is in a substantially upright position.

Other aspects and embodiments of the invention will become apparent in light of the following description and drawings.

### BRIEF DESCRIPTION OF THE DRAWINGS

FIG. 1 is a pictorial view of a user inserting an eye drop into the eye using a conventional eye dropper.

FIG. 2 is a side view of a system for administering medication according to an embodiment of the invention.

FIG. 3 is a schematic illustration of a system for administering medication according to another embodiment of the invention.

FIG. 4 is a schematic illustration of a system for administering medication according to yet another embodiment of the invention.

FIG. 5 is a schematic illustration of a system for administering medication according to still another embodiment of the invention.

It should be understood that the invention is not limited in its application to the details of construction and the arrangements of the components set forth in the following description or illustrated in the above-described drawings. The invention is capable of other embodiments and of being practiced or being carried out in various ways. Also, it is to be understood that the phraseology and terminology used herein is for the purpose of description and should be not regarded as limited.

### DETAILED DESCRIPTION

Described herein are systems and methods for administering medication to an eye with the system substantially in an upright position. The system includes a siphon tube in fluid communication with a medication reservoir, a dispensing nozzle connected to the siphon tube, and a shield coupled to the dispensing nozzle for movement relative thereto between an opened position and a closed position. The siphon tube defines a longitudinal axis extending along a substantially vertical direction when the siphon tube is in the upright position. The dispensing nozzle extends at an angle offset from the longitudinal axis when the shield is in the opened position.

### 1. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

Section headings as used in this section and the entire disclosure herein are not intended to be limiting.

For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the use of the term “about” indicates that values slightly outside the cited values, namely, plus or minus 10%. Such values are thus encompassed by the scope of the claims reciting the terms “about” and “approximately.”

The terms “administer,” “administering,” “administered” or “administration” refer to providing a compound or a pharmaceutical composition (e.g., one described herein), to a subject or patient.

As used herein, the term “subject” is intended to include human and non-human animals. Exemplary human subjects include a human patient having a disorder, e.g., glaucoma, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

### 2. SYSTEM FOR ADMINISTERING MEDICATION TO AN EYE

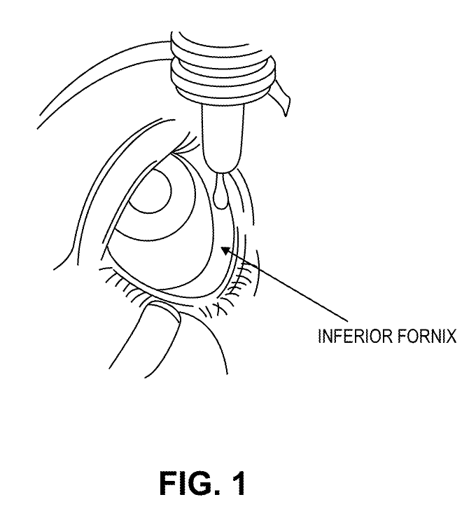

Conventional eye drop bottles require inversion for administering medication to an eye, which may pose a difficulty for patients. As illustrated in FIG. 1, to dispense a drop from a conventional eye drop bottle, the bottle is inverted toward a direction substantially perpendicular to the plane of the cornea, so that the drop can fall downwardly and onto the cornea or into the cul-de-sac. The conventional design may require the patient to either recline the head or lie flat such that the plane of the cornea is horizontal. However, glaucoma patients (e.g., with arthritis) may have difficulty with this positioning. The systems disclosed herein can advantageously reduce the hand strength and dexterity required for administering medication to an eye, and make it easy to deliver medication while holding the bottle still. Moreover, the eye drop delivery can be made quick, reducing the chance for blinking. Furthermore, the user can be put in control of drop delivery timing.

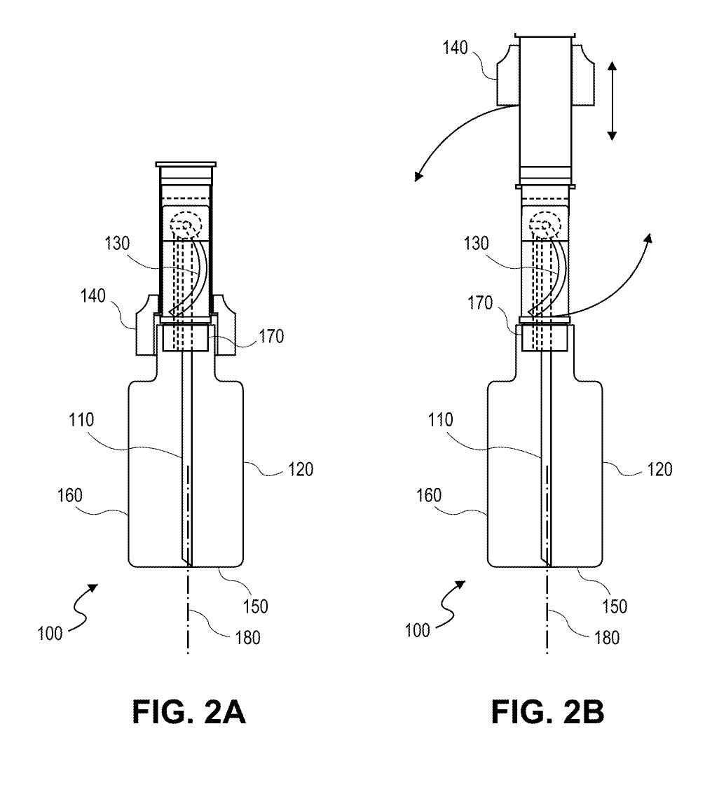

Referring to FIG. 2, a system 100 for administering medication to an eye includes a siphon tube or dip tube 110 in fluid communication with a medication reservoir, container, or bottle 120, a dispensing nozzle or tube 130 connected to the siphon tube 110, and a shield or cap 140 coupled to the dispensing nozzle 130 for movement relative thereto between an opened position and a closed position. The medication reservoir 120 has a base 150 and one or more walls 160 joined to and extending from the base. The wall 160, at its distal end spaced from the base 150, defines an opening 170. When the medication reservoir 120 is in an upright position, the opening 170 is positioned substantially above the base 150. In the illustrated embodiment, the shield 140 is coupled to the opening 170 (e.g., via threads). The shield 140 can inhibit environmental elements such as dust or debris from coating, contacting, or contaminating the dispensing nozzle 130. The siphon tube 110 defines a longitudinal axis 180 extending along a substantially vertical direction when the siphon tube 110 is in an upright position. The dispensing nozzle 130 extends at a substantially right angle from the longitudinal axis 150 when the shield 140 is in the opened position. The nozzle 130 and the angle from the longitudinal axis 150 to the dispensing nozzle 130 are so dimensioned as to facilitate accessing the eye when the siphon tube 110 is substantially in the upright position.

The system 100 according to this invention may be made of any material having sufficient pliability and elasticity. Such materials are known in the art and include, but are not limited to, plastics such as polyurethane, Loctite® 3921 UV adhesive, Elastosil® LR 3043/50 silicone, polyetheretherketone, and silicone. In some embodiments, the system 100 can be made at least in part by stainless steel.

With continued reference to FIG. 2, to administer a drop or dose of the medication, the user unscrews the shield 140 (e.g., upwardly in FIG. 2) to an opened position. The dispensing nozzle 130 is rotated counterclockwise in FIG. 2B such that it extends at a substantially right angle from the longitudinal axis 180. As used herein, the terms “upwardly,” “downwardly,” “top,” “bottom,” “clockwise,” “counterclockwise,” and other directional terms are not intended to require any particular orientation, but are instead used for purposes of description only. The right angle between the dispensing nozzle 130 and the longitudinal axis 180 can allow the user to express a drop or dose into the inferior fornix while maintaining the head in an upright position, for example by squeezing the medication reservoir 120.

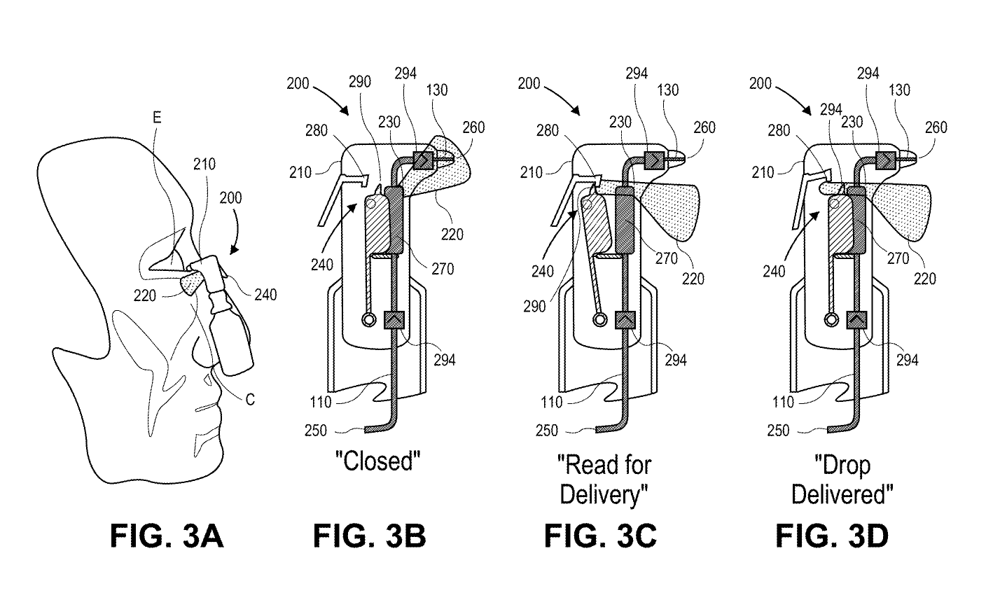

FIG. 3 illustrates the system 200 for administering medication according to another embodiment of the invention. This embodiment employs much of the same structure and has many of the same features as the embodiment of the system 100 described above in connection with FIG. 2. Accordingly, the following description focuses primarily upon the structure and features that are different than the embodiment described above in connection with FIG. 2. Reference should be made to the description above in connection with FIG. 2 for additional information regarding the structure and features and possible alternatives to the structure and features of the system 200 illustrated in FIG. 3 and described below. Structure and features of the embodiment shown in FIG. 3 that correspond to structure and features of the embodiment of FIG. 2 are designated hereinafter with like reference numbers.

The system 200 in this embodiment includes a housing 210, a shield 220 coupled thereto at a pivot joint 230, and an actuator 240 for activating a dosing mechanism for medication. The shield 220 is rotatable at the pivot joint 230 between a closed position (see FIG. 3B) and an opened position (see FIG. 3C), around an axis extending into and out of the plane in FIGS. 3B-3D. In the illustrated embodiment, the shield 220 extends upwardly in the closed position and substantially horizontally in the opened position. The illustrated shield 220 is thus rotated or flipped downwardly (i.e., clockwise in FIGS. 3B-3D) from the closed position to the opened position. In other embodiments, the shield 220 may be rotated or flipped upwardly from the closed position to the opened position. In further embodiments, the shield 220 may be loaded or biased by suitable mechanisms. With continued reference to FIG. 3, in use, the user rotates or flips the shield 220 from the closed position to the opened position, and places the shield 220 against a facial structure such as a cheek C. In this regard, the shield 220 can be used as a cheek rest, and a distance between the eye E and the dispensing nozzle 130 can therefore be controlled. Moreover, potential contamination caused by the dispensing nozzle 130 touching facial skin or an ocular surface may be reduced.

In the illustrated embodiment, the shield 220 is not removable from the housing 210, and therefore the shield 220 will not be lost or thrown away after it is moved from the closed position to the opened position. In other embodiments, however, the shield 220 may be removable from the housing 210, for example upon being opened. In some embodiments, the shield 220 may be made from medical grade plastic such as silicone or thermoplastic elastomer for compatibility to a range of facial structures. In other embodiments, the shield 220 may be made from other materials.

As illustrated in FIG. 3B, the siphon tube 110 and dispensing nozzle 130 collectively define an inlet end 250 and an outlet end 260 opposite the inlet end 250. A pressure chamber 270 is connected between the inlet and outlet ends 250, 260. In the illustrated embodiment, the dispensing nozzle 130 defines a first inner diameter, and the pressure chamber 270 defines a second inner diameter. The second inner diameter is greater than the second inner diameter. In some embodiments, one or both of the dispensing nozzle 130 and pressure chamber 270 may have a cross-sectional shape other than circular (e.g., oval, square, rectangular, or other regular or irregular shapes) in which cases the outermost diameters as used herein may include dimensions other than a diameter, for example the lengths of major axes or the cross-sectional area of the dispensing nozzle 130 and pressure chamber 270. The different inner diameters can facilitate giving the medication an extra velocity to project it outward when pressure (e.g., negative pressure) is applied to the chamber 270, as explained below.

In the illustrated embodiment, the actuator 240 includes a detent or button 280 and a corresponding catch mechanism 290 connected to the pressure chamber 270. When the shield 220 is in the closed position, the detent 280 is in an unlocked position relative to the catch mechanism 290. Referring to FIG. 3C, in response to the shield 220 being opened, the detent 280 and the corresponding catch mechanism 290 are moved into a locking position. Pivoting the shield 220 clockwise or downwardly in FIGS. 3B-3D moves the catch mechanism 290 slightly counterclockwise or to the left in FIGS. 3B-3D. The detent 280 thereby contacts and locks or cocks the catch mechanism 290 in a “ready for delivery” state. When the user depresses the detent 280 relative to the housing 210, the catch mechanism 290 applies pressure to the pressure chamber 270, thereby delivering the medication through the dispensing nozzle 130. Referring also to FIG. 3D, when the delivery of the medication is complete, the detent 280 is released out of the locking position relative to the catch mechanism 290. The user can then move the shield 220 from the opened position to the closed position, thereby completing a cycle. In some embodiments, the actuator 240 may accomplish activating a dosing mechanism by means of other mechanical, hydraulic, pneumatic, or electric systems depending upon the capabilities and configuration of the actuator 240.

The system 200 optionally includes one or more check valves 294. The check valves 294 can facilitate moving the medication in one direction only and/or toward a predetermined direction. The check valves 294 may be made from medical grade plastic from other materials. In some embodiments, a filter or screen (not shown) may be coupled adjacent the inlet end 250. The filter may be configured to substantially prevent contamination of the medication reservoir 120, and to prevent the medication from leaking out.

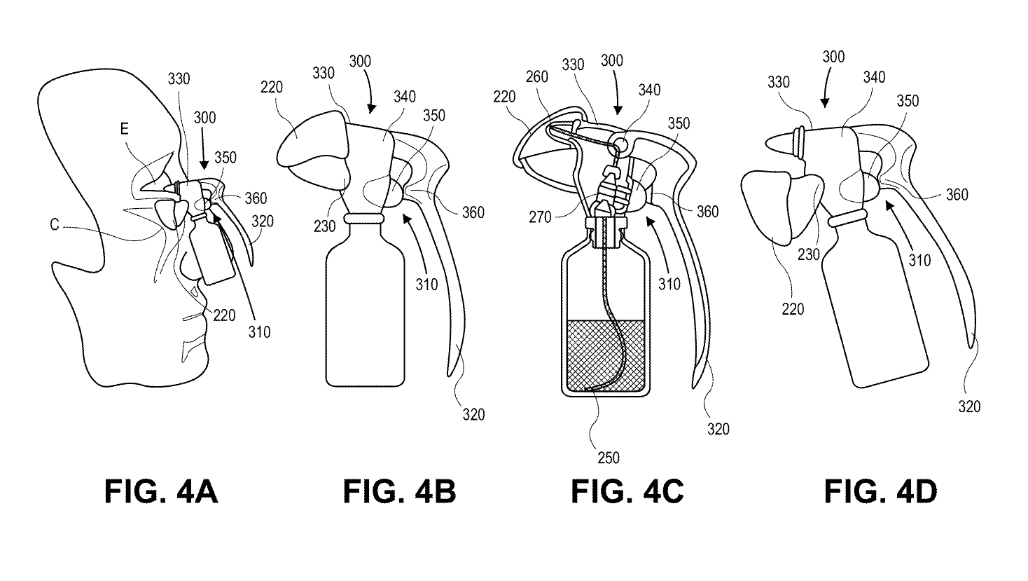

FIG. 4 illustrates a system 300 for administering medication according to another embodiment of the invention. This embodiment employs much of the same structure and has many of the same features as the embodiment of the systems 100 and 200 described above in connection with FIGS. 2 and 3, respectively. Accordingly, the following description focuses primarily upon the structure and features that are different than the embodiments described above in connection with FIGS. 2 and 3. Reference should be made to the description above in connection with FIGS. 2 and 3 for additional information regarding the structure and features and possible alternatives to the structure and features of the system 300 illustrated in FIG. 4 and described below. Structure and features of the embodiments shown in FIG. 4 that correspond to structure and features of the embodiments of FIGS. 2 and 3 are designated hereinafter with like reference numbers.

The actuator 310 in this embodiment includes a handle 320 coupled to the housing 330 at a second pivot joint 340, and a button or switch 350 coupled to the housing 330. The button 350 is spring-loaded or biased by any other suitable mechanisms. The handle 320 has an actuating portion 360 that cooperates with the button 350 to deliver the medication. In some embodiments, the handle 320 may be ergonomic to use. When the user rotates the handle 320 clockwise in FIGS. 4B-4D from a resting position toward an actuating position, the actuating portion 360 depresses the button 350 against a bias, thereby applying pressure to the pressure chamber 270, and delivering a predetermined amount of medication through the dispensing nozzle 130 in response to the movement in the handle 320. In the illustrated embodiment, the button 350 travels a predetermined distance that is not dependent on the force exerted by the patient. Thus, the patient can consistently apply pressure to the chamber 270, for example resulting in a single drop of medication delivered, and thereby reducing potential waste and overdosing or underdosing.

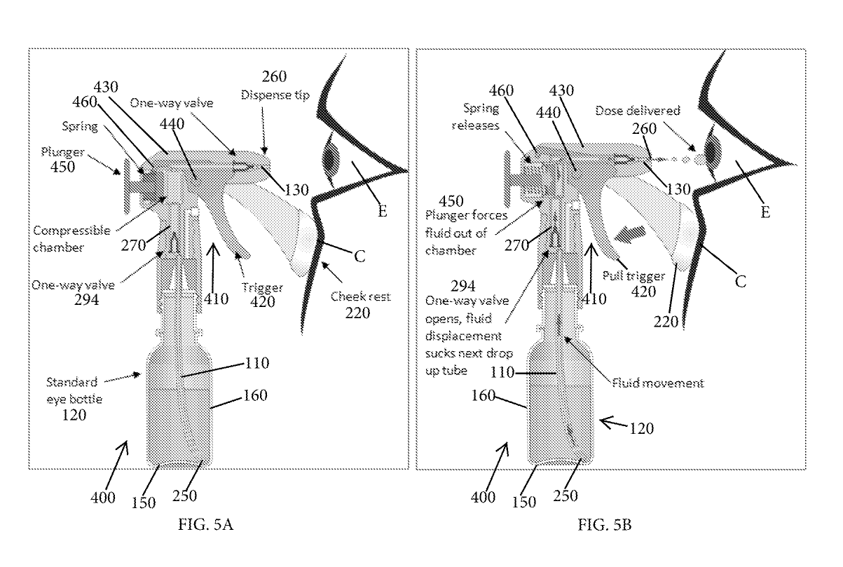

FIG. 5 illustrates a system 400 for administering medication according to another embodiment of the invention. This embodiment employs much of the same structure and has many of the same features as the embodiment of the systems 100, 200, and 300 described above in connection with FIGS. 2-4, respectively. Accordingly, the following description focuses primarily upon the structure and features that are different than the embodiments described above in connection with FIGS. 2-4. Reference should be made to the description above in connection with FIGS. 2-4 for additional information regarding the structure and features and possible alternatives to the structure and features of the system 400 illustrated in FIG. 5 and described below. Structure and features of the embodiments shown in FIG. 5 that correspond to structure and features of the embodiments of FIG. 2-4 are designated hereinafter with like reference numbers.

The actuator 410 in this embodiment includes a trigger or handle 420 coupled to the housing 430 at a second pivot joint 440 adjacent the shield 220, and a plunger or piston 450 coupled to the housing 430 opposite the trigger 420. The plunger 450 is spring-loaded or biased by any other suitable mechanisms. The trigger 420 includes a detent 460 that locks or engages the plunger 450 and keeps the plunger 450 away from the pressure chamber 270. When the user rotates or pulls the trigger 420 clockwise in FIG. 5 from a resting position (see FIG. 5A) toward an actuating position (see FIG. 5B), the detent 460 on the trigger 420 releases or unlocks the plunger 450, thereby allowing the plunger 450 to move toward the pressure chamber 270 and apply a pressure to the pressure chamber 270. A predetermined amount of medication is therefore delivered through the dispensing nozzle 130 in response to the movement in the trigger 420.

### 3. METHODS OF USE

The present invention is also directed to a method for administering medication to an eye of a user. In use, the user opens the shield 140, 220 coupled to the dispensing nozzle 130 of the system 100, 200, 300 for administering medication. The shield 140, 220 is placed against a facial structure (e.g., cheek C) of the user, a dosing mechanism is activated, and the user's eye E is contacted with the medication, all when the system 100, 200, 300, 400 is in a substantially upright position. In some embodiments, opening the shield 140, 220 includes pivotally moving the shield 140, 220. In further embodiments, activating the dosing mechanism includes pivotally moving the handle 320, 420.

### Example 1

An embodiment of a prototype system was tested in a group of patients who regularly use eye drops. Fifteen subjects were recruited to test the prototype system. Each subject attempted to instill a drop of saline in his or her eye with the prototype system and separately with a conventional eye drop bottle. Each subject was allowed three trials of each system (both prototype and conventional), alternating between the two systems and between the two eyes. The first system used and the first eye into which the drop was instilled were randomly assigned. A trained observer recorded, for each trial, (1) how long it took for the subject to instill the drop into the eye, (2) if the drop was successfully instilled, (3) if excess drops ran down the cheek, and (4) if the subject contaminated the outlet by touching the skin or ocular surface. Subjects were also queried as to their subject impression of the prototype system.

Results of the study showed that the prototype system allowing upright drop administration resulted in less contamination of the outlet. Subjects using the prototype system took longer to instill into the eye compared to the conventional system (Trial 3, average time 24.3±12.7 seconds versus 15.8±6.4 seconds, respectively, p=0.016). However, the time to instillation improved with each successive trial of the prototype system (average time 38.2±24.3 seconds for Trial 1 and 24.3±12.7 seconds for Trial 3). There were no significant differences between the systems regarding the proportion of drops successfully instilled into the eye for any of the three trials. Although the proportion of attempts during which excess drops was greater in each trial for the prototype system versus the conventional system, the differences were not significant. For all trials, the proportion of attempts during which the system outlet was contaminated was greater for the conventional system than for the prototype system, and for two trials, the difference was statistically significant (Trial 1, in conventional system, 47% of attempts resulted in contamination; in prototype system, 13%, of attempts resulted in contamination; p=0.025. Trial 3, in conventional system, 38% of attempts resulted in contamination; in prototype system, 8% of attempts resulted in contamination; p=0.025).

### Example 2

The effectiveness and ease-of-use of the systems disclosed herein will be tested in a population of patients with glaucoma: (1) an observational study of the systems disclosed herein will be conducted in a group of patients with glaucoma; and (2) the conventional system will be compared to the systems disclosed herein for (a) successfully instilling the drop into the eye (primary outcome), (b) time to instill a drop into the eye, (c) expression of excess drops, and (d) avoiding contamination of the bottle by touching the skim or ocular surface.

Sample size estimates are derived from Example 1. In particular, sample sizes will be calculated based on two outcomes: (1) the ability to instill a drop into the eye, deemed an important outcome, and (2) the ability to instill a drop into the eye without contaminating the bottle, the outcome for which the previously tested prototype system differed most from the conventional system. The results of the third trial will be used for estimates, as the timing results suggested a learning curve for the subjects using the prototype system. Calculations will be performed for both right and left eyes, and the final sample size estimate will be the average of the two.

Based on results for the right eye for the outcome “ability to instill an eyedrop,” a sample size of 113 pairs will have 90% power to detect a difference in proportions of 0.077 when the proportion of discordant pairs is expected to be 0.087 and the method of analysis is a McNemar's test of equality of paired proportions with a 0.050 two-sided significance level. Based on results for the left eye for the outcome “ability to instill an eyedrop,” a sample size of 50 pairs will have 90% power to detect a difference in proportions of 0.154 when the proportion of discordant pairs is expected to be 0.164 and the method of analysis is a McNemar's test of equality of paired proportions with a 0.050 two-sided significance level. Based on results for the right eye for the outcome “ability to instill an eyedrop without contamination,” a sample size of 36 pairs will have 90% power to detect a difference in proportions of 0.308 when the proportion of discordant pairs is expected to be 0.408 and the method of analysis is a McNemar's test of equality of paired proportions with a 0.050 two-sided significance level. Based on results for the left eye for the outcome “ability to instill an eyedrop without contamination,” a sample size of 50 pairs will have 90% power to detect a difference in proportions of 0.154 when the proportion of discordant pairs is expected to be 0.164 and the method of analysis is a McNemar's test of equality of paired proportions with a 0.050 two-sided significance level.

As each subject will test each bottle, each subject will be considered a “pair” for these calculations. As such, to accomplish the goals of detecting a difference in both the ability to instill a drop and the ability to do so without contamination, a total of 82 subjects will be enrolled. Potentially eligible subjects will be detected by review of electronic medical record, and approached during their regularly scheduled clinic visit. Possible exclusion criteria include, but are not limited to, intraocular surgery in the past 1 month as recent operations may increase the risk of infection and visual acuity <20/400 in the better-seeing eye, as severe vision loss may confound outcome assessment.

Eligible subjects who give informed consent will view a video describing proper use of both the traditional and novel eye drop bottles. The study team will create a brief video describing an eye drop instillation technique for the novel bottle. Subjects will view the video to ensure that each subject receives the same instruction. The study coordinator will inform subjects that he or she cannot answer questions about technique but can replay the video as needed. Subjects will be asked to instill an eye drop into the eye with each the conventional system and the systems disclosed herein. Each subject will be asked to instill a drop from each system into each eye, three times. The first eye and the first system used will be determined randomly.

The study coordinator will instruct the subjects as to the use of the traditional system and refer them to the video for instruction on the systems disclosed herein. Following the video, subjects will be given a plastic eye drop bottle filled with sterile saline, similar in size and shape to most glaucoma medications as well as the systems disclosed herein, also filled with sterile saline. The study coordinator will read the following script to the subject: “These two bottles are filled with sterile saline only—no medication is present. This bottle [indicating the conventional system] is similar in design to most glaucoma eye drop bottles. I will refer to it as the traditional bottle. This bottle [indicating the systems disclosed herein] is the new design. I will refer to it as the new bottle. Using the traditional bottle, I would like you place a drop of the saline into your eye just as you would with your glaucoma medication at home. Using the new bottle, I would like you to place a drop of saline into your eye as described in the video. I will instruct as to which bottle to use when and into which eye to instill the drop. I will be observing you while you do this.”

The study coordinator will score each trial for time, instillation, waste, and contamination. For each trial, the study coordinator will record (1) the time from opening the bottle to eye drop instillation, (2) whether or not the drop was successfully instilled into the cornea or into the cul-de-sac, (3) if excess drops were expressed, and (4) if the tip of the bottle was contaminated by touching the skin or ocular surface.

Descriptive statistics for each outcome will be derived. Descriptive statistics will computed, separately by Trial (1, 2, 3), eye (OD, OS) and bottle (traditional, novel). For the outcome variable, time to instill the drops, the means, standard deviations, minimums, medians, and maxima will be computed. Additionally, plots of the mean values will be produced. For the other outcome variables (drop was instilled, excess drops were expressed, and bottle tip was contaminated), frequencies and percentages will be computed. In addition, plots of the percentages will be produced.

For each outcome, the traditional bottle will be compared to the devices disclosed herein. The significance of the difference between bottles for time to instillation of drops will be assessed using the Wilcoxon signed rank test of median difference equal to zero for paired data. The significance of the difference between bottles in the proportions of drops instilled, excess drops wasted, and contaminated will be assessed using the McNemar's test for paired data.

Following the last trial, each subject will be asked a serious of open-ended questions and the coordinator will audiorecord the responses. Suggested questions may include, but are not limited to, the following:

“What is your overall impression of the new bottle?”

“Was it easier or harder to use than the traditional bottle?

“In what way?”

“What suggestions would you make to improve upon the design of the new bottle?”

“If the new bottle were available for your glaucoma medication, would you use it instead of the traditional bottle?”

Audio-recordings will be transcribed verbatim by a transcription service. The study coordinator will also take written notes to capture emotional responses that might not be evident in transcription.

Qualitative data analysis software (ATLAS.ti 5.2) will be used to code the study data. Using ATLAS.ti, reports of all text segments for each code will be generated. The degree to which the construct appears in the data and the degree to which the construct positively or negatively reflects the devices disclosed herein will be assessed.

It is understood that the invention may embody other specific forms without departing from the spirit or central characteristics thereof. The disclosure of aspects and embodiments, therefore, are to be considered as illustrative and not restrictive. While specific embodiments have been illustrated and described, other modifications may be made without significantly departing from the spirit of the invention.

Various features and advantages of the invention are set forth in the following claims.

What is claimed is:

1. A system for administering medication to an eye, the system comprising:
a siphon tube in fluid communication with a medication reservoir, the siphon tube defining a longitudinal axis extending along a substantially vertical direction when the siphon tube is in an upright position;
a dispensing nozzle connected to the siphon tube; and
a shield coupled to the dispensing nozzle for movement relative thereto between an opened position and a closed position, wherein the dispensing nozzle extends at an angle offset from the longitudinal axis when the cover is in the opened position, and wherein the nozzle and the angle are so dimensioned as to facilitate accessing the eye when the siphon tube is substantially in the upright position.

2. The system of claim 1, wherein the siphon tube and dispensing nozzle collective define an inlet end and an outlet end opposite the inlet end, wherein a chamber is connected between the inlet and outlet ends, wherein the dispensing nozzle defines a first inner diameter, wherein the chamber defines a second diameter, and wherein the second inner diameter is greater than the first inner diameter.

3. The system of claim 1, wherein the siphon tube and dispensing nozzle collectively define an inlet end and an outlet end opposite the inlet end, wherein the system further comprises a chamber connected between the inlet and outlet ends, a detent and a catch mechanism, at least one of which is connected to the chamber, wherein the detent and the catch mechanism are moved into a locking position in response to the shield being opened, and wherein the detent and catch cooperate to apply a pressure to the chamber, thereby delivering the medication.

4. The system of claim 3, wherein the detent and catch mechanism are released out of the locking position when delivery of the medication is complete.

5. The system of claim 1, further comprising a housing, and wherein the shield is coupled to the housing at a pivot joint.

6. The system of claim 1, further comprising a housing, a handle coupled to the hosing for movement relative thereto, and a switch coupled to the housing, wherein the handle and switch cooperate to deliver a predetermined amount of the medication in response to a movement in the handle.

7. The system of claim 6, wherein the handle is coupled to the housing at a pivot joint.

8. The system of claim 6, wherein the movement is associated with a movement force, and wherein the predetermined amount of the medication is substantially independent of the movement force.

9. The system of claim 1, wherein the shield is made of medical grade plastic.

10. A system for administering medication to an eye, the system comprising:
a container having a base and an opening positioned substantially above the base when the container is in an upright position;
a siphon tube in fluid communication with the container, the siphon tube defining a longitudinal axis;
a dispensing nozzle connected to the siphon tube; and
a shield coupled to the dispensing nozzle for movement relative thereto between an opened position and a closed position, wherein the dispensing nozzle extends at an angle offset from the longitudinal axis when the shield is in the opened position, and wherein the nozzle and the angle are so dimensioned as to facilitate accessing the eye when the container is substantially in the upright position.

11. The system of claim 10, wherein the siphon tube and dispensing nozzle collectively define an inlet end and an outlet end opposite the inlet end, wherein a chamber is connected between the inlet and outlet ends, wherein the dispensing nozzle defines a first inner diameter, wherein the chamber defines a second diameter, and wherein second inner diameter is greater than the first inner diameter.

12. The system of claim 10, wherein the siphon tube and dispensing nozzle collectively define an inlet end and an outlet end opposite the inlet end, the system further comprising a chamber connected between the inlet and outlet ends, a detent and a catch mechanism, at least one of which is connected to the chamber, wherein the detent and the catch mechanism are moved into a locking position in response to the shield being opened, and wherein the detent and catch cooperate to apply a pressure to the chamber, thereby delivering the medication.

13. The system of claim 12, wherein the detent and catch mechanism are released out of the locking position when delivery of the medication is complete.

14. The system of claim 10, further comprising a housing removably coupled to the container, and wherein the shield is coupled to the housing at a pivot joint.

15. The system of claim 10, further comprising a housing removably coupled to the container, a handle coupled to the hosing for movement relative thereto, and a switch coupled to the housing, wherein the handle and switch cooperate to deliver a predetermined amount of the medication in response to a movement in the handle.

16. The system of claim 15, wherein the handle is coupled to the housing at a pivot joint.

17. The system of claim 15, wherein the movement is associated with a movement force, and wherein the predetermined amount of the medication is substantially independent of the movement force.

18. A method for administering medication to an eye of a user, the method comprising:
opening a shield coupled to a dispensing nozzle of a system for administering medication;
placing the shield against a facial structure of the user;
activating a dosing mechanism; and
contacting the user's eye with the medication, all when the system is in a substantially upright position.

19. The method of claim 18, wherein opening the shield includes pivotally moving the shield.

20. The method of claim 18, wherein activating the dosing mechanism includes pivotally moving a handle.
